(19) United States
(12) Patent Application Publication
(10) Pub. No.: US 2025/0248830 A1
(43) Pub. Date: Aug. 7, 2025

(54) METHOD AND DEVICE FOR CORRECTING A DEFORMED HEART VALVE FRAME

(71) Applicant: Medical Equipment Design Innovation, Inc., Irvine, CA (US)

(72) Inventor: Pham Cong Lo, Rancho Mission Viejo, CA (US)

(73) Assignee: Medical Equipment Design Innovation, Inc., Irvine, CA (US)

(21) Appl. No.: 18/434,324

(22) Filed: Feb. 6, 2024

(51) Int. Cl. A61F 2/90; A61F 2/24; A61F 2/844; A61F 2/966; A61L 31/02

(52) U.S. Cl. CPC A61F 2/90; A61F 2/2412; A61F 2/844; A61F 2/966; A61L 31/022

(57) ABSTRACT

A method is provided for repairing an implanted heart valve prosthesis, wherein the heart valve prosthesis has a deformed skirt section that is positioned at a native valve annulus, and a valve section which has a plurality of leaflets. An assist stent device is provided having an annular frame surrounded by an annular tissue assembly, wherein the annular frame has a generally cylindrical body made of cobalt-chromium having a plurality of rows of cells. The assist stent device is delivered to the native valve annulus, and aligned inside the skirt section. The assist stent device is then expanded until the assist stent device engages the skirt section and secures the skirt section against the native valve annulus.

### BACKGROUND OF THE INVENTION

### 1. Field of the Invention

The present invention relates to the field of transcatheter aortic heart valve replacement, and in particular, to a method and device for correcting a deformed frame for a transcatheter heart valve prosthesis that has been deployed at the aortic annulus.

### 2. Description of the Prior Art

Transcatheter heart valves are now commonly used to treat valve disease. These heart valves are typically made of a frame that supports a prosthetic heart valve having leaflets that are usually made of bovine pericardium or porcine pericardium. There are a few types of transcatheter aortic heart valves that are available in the market.

One common type has a frame that is made of a shape memory material such as nickel-titanium. The frame on this type of heart valve is self-expanding, but because the radial force of a nickel-titanium frame is low and because nickel-titanium is a flexible material, the frame can easily migrate from its implanted position, or can be easily deformed due to any number of conditions.

When a transcatheter heart valve prosthesis with a nickel-titanium frame is initially deployed at the aortic annulus of a patient, there is a possibility that the frame may be deformed because the radial force of the nickel-titanium frame material is not strong enough (i.e., it is usually between 15 newton/bls and 25 newton/bls) to ensure a uniform cylindrical expansion. A deformed nickel-titanium frame implanted at the aortic annulus can result in defective performance of the valve leaflets and paravavular leaked at the skirt section. Specifically, the deformed frame will cause the leaflets of the heart valve prosthesis to be mismatched, thereby creating a channel that results in a leak. Most of physicians will try to use the balloon to re-expand the nickel-titanium frame in an effort to reshape frame, but the frame can easily deform again after implantation as nickel-titanium is a shape memory material.

Thus, there remains a need to correct or repair a deformed nickel-titanium frame for a transcatheter heart valve prosthesis that has been implanted at the aortic annulus.

### SUMMARY OF THE DISCLOSURE

It is an object of the present invention to provide a device for correcting or repairing a deformed nickel-titanium frame for a transcatheter heart valve prosthesis that has been implanted at the aortic annulus.

It is another object of the present invention to provide a method for correcting or repairing a deformed nickel-titanium frame for a transcatheter heart valve prosthesis that has been implanted at the aortic annulus.

To meet the objectives of the present invention, there is provided a method for repairing an implanted heart valve prosthesis, wherein the heart valve prosthesis has a deformed skirt section that is positioned at a native valve annulus, and a valve section which has a plurality of leaflets. An assist stent device is provided having an annular frame surrounded by an annular tissue assembly, wherein the annular frame has a generally cylindrical body made of cobalt chromium having a plurality of rows of cells. The assist stent device is delivered to the native valve annulus, and aligned inside the skirt section. The assist stent device is then expanded until the assist stent device engages the skirt section and secures the skirt section against the native valve annulus. Since the assist stent device is made from a much stiffer material, such as cobalt chromium, it is able to re-shape and support the deformed skirt section at the location of the aortic annulus and high calcification leaflet.

### BRIEF DESCRIPTION OF THE DRAWINGS

FIG. 1 is a perspective view of an assist stent device according to one embodiment of the present invention.

FIG. 2 is a side view of the assist stent device of FIG. 1.

FIG. 3 is a perspective view of the frame for the assist stent device of FIG. 1.

FIG. 4 is a side view of the frame of FIG. 3.

FIG. 5 is a side view of the tissue assembly for the assist stent device of FIG. 1.

FIG. 6 is a schematic view of a delivery system according to the present invention showing the assist stent device of FIG. 1 compressed on the balloon.

FIG. 7 is a schematic view of the delivery system of FIG. 6 shown with the capsule being withdrawn to expose the assist stent device of FIG. 1.

FIG. 8 is a schematic view of the delivery system of FIG. 6 shown with the balloon expanded to expand the assist stent device of FIG. 1.

FIG. 9 illustrates the delivery system and assist stent device of FIG. 6 being positioned within a transcatheter aortic heart valve prosthesis that has a deformed frame.

FIG. 10 illustrates the balloon of the delivery system being expanded with the delivery system and assist stent device of FIG. 6 being positioned within the transcatheter aortic heart valve prosthesis of FIG. 9.

FIG. 11 illustrates the balloon of the delivery system being deflated with the delivery system and assist stent device of FIG. 6 being positioned within the™ transcatheter aortic heart valve prosthesis of FIG. 9.

FIG. 12 illustrates an aortic valve prosthesis that is usually implanted at a native aortic annulus position.

FIG. 13 illustrates the assist stent device implanted at the aortic valve prosthesis of FIG. 12.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention provides an assist stent device 100 that can be used for correcting or repairing a deformed nickel-titanium frame for a transcatheter heart valve prosthesis that has previously been implanted at the aortic annulus at a human heart, including a method for such correction and repair. Specifically, the assist stent device 100 has a frame 200 that is provided in a more rigid material, such as cobalt chromium, than the existing nickel-titanium frame, so that it can assist the original nickel-titanium frame in securing the existing prosthesis to the native annulus.

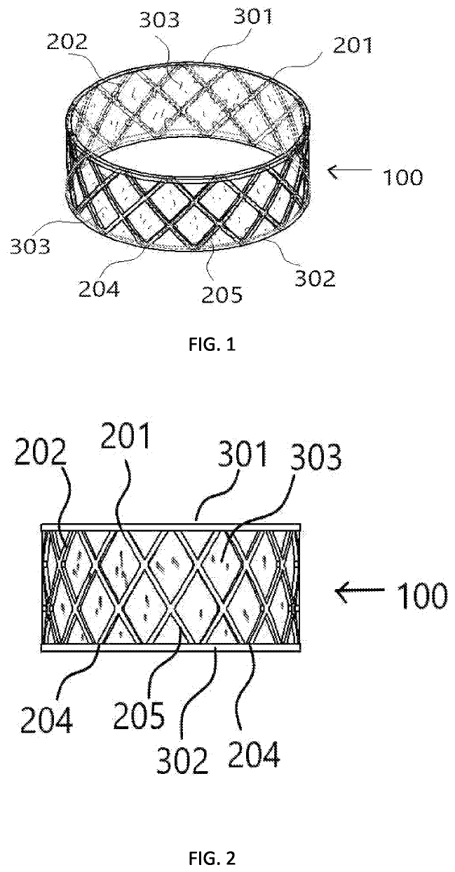

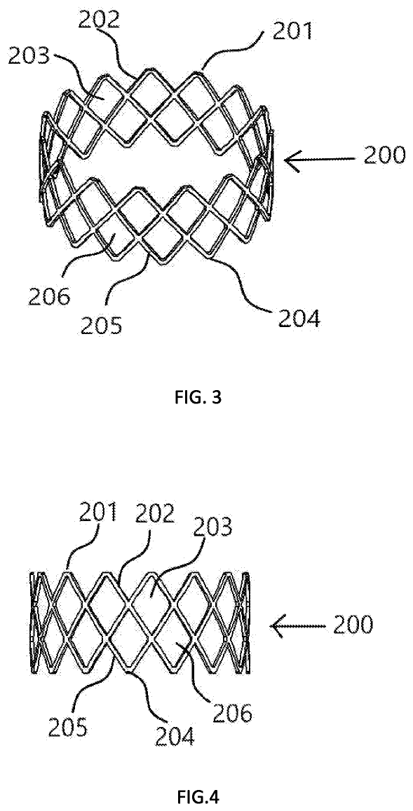

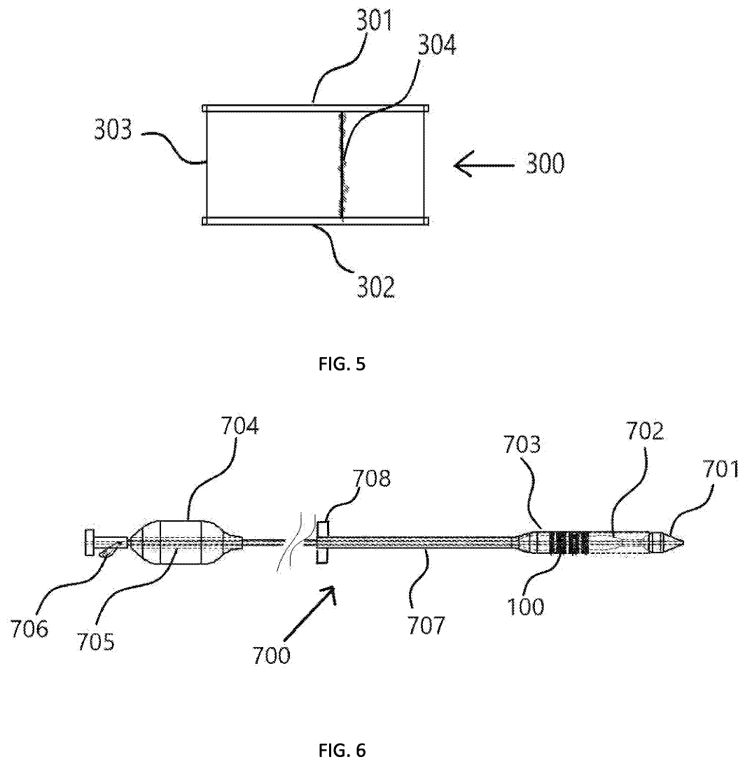

FIGS. 1-5 show the assist stent device 100. FIGS. 3 and 4 show the frame 200 alone, while FIG. 5 shows the tissue assembly 300 alone.

The assist stent device 100 has an annular frame 200 surrounded by an annular tissue assembly 300. The frame 200 has a generally cylindrical body having two rows of cells, with a first row 203 and a second row 206. Each cell in the two rows 203 and 206 of cells is made up of four struts (e.g., first struts 202 and second struts 205) that define a diamond shape, and with a plurality of spaced-apart first tips 201 defined by the vertices of two adjacent first struts 202, and a plurality of spaced-apart second tips 204 defined by the vertices of two adjacent second struts 205. The tips 201 and 204 define peaks for a zig-zag configuration of peaks and valleys for each end of the frame 200. The frame 200 can have the same diameter throughout.

The tissue assembly 300 has an annular tissue 303 having a first annular end 301 is that is adapted to be attached (e.g., by stitching) to the first tips 201, and an opposing second annular end 302 that is adapted to be attached (e.g., by stitching) to the second tips 204. A stitch line 304 can be used to stitch or join the ends of a flat piece of tissue 303 to form the annular shape of the tissue assembly 300.

The frame 200 is preferably made from a rigid material, such as cobalt-chromium. Specifically, the frame 200 can be made by cobalt-chrome small tubing (e.g., 7 mm). The design can be laser-cut on the tubing and expanded like a shaped cylinder with a 23 mm, 26 mm, 29 mm, or 32 mm diameter.

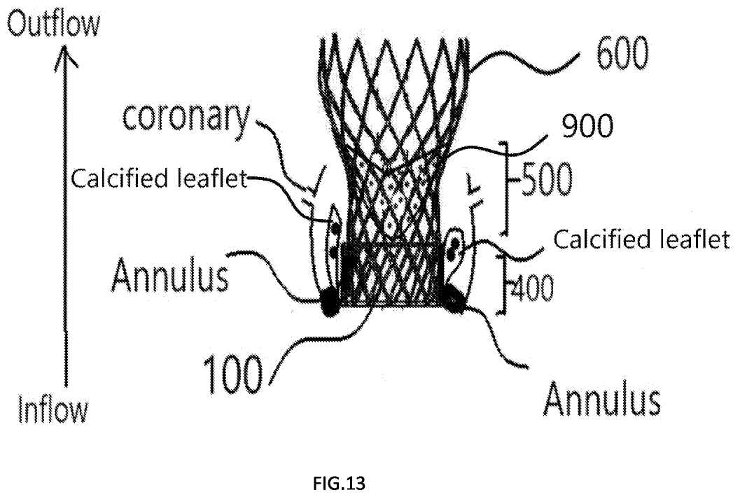

Referring to FIG. 12, the aortic valve prosthesis 600 can have a nickel-titanium skirt section 400 that is positioned at the aortic annulus with the calcified native human leaflets, and a valve section 500 where the leaflets 900 are positioned. FIG. 12 shows the prosthesis 600 can have a skirt section 400 that is deformed due to high calcification, and FIG. 13 shows how an implanted the assist stent device 100 is used to correct the deformity in the skirt section 400.

FIGS. 6-11 illustrate a delivery system 700 that is adapted for use in delivering the assist stent device 100 to an aortic annulus which has a previously implanted aortic valve prosthesis 600, and deploying it inside the aortic valve prosthesis to support the underlying nickel-titanium frame. The delivery system 700 includes a balloon catheter and a sheath assembly that is sized and configured to ensheath and release the assist stent device 100.

The balloon catheter has a shaft 705 that extends from a T-junction 706 (which acts as a flow connector) to a tapered distal tip 701. An inflatable balloon 702 is provided on the shaft 705 adjacent the tapered distal tip 701. The shaft 705 acts as a flow tube that delivers inflation fluid to the balloon 702. A handle 704 is provided adjacent the T-junction 706. The assist stent device 100 is positioned at the location of the balloon 702, and surrounding the balloon 702.

The sheath assembly has a hollow shaft 707 with a lumen that is sized to receive the shaft 705 of the balloon catheter. A caplock 708 is provided at the proximal end of the hollow shaft 707 and functions to move a capsule 703 distally (to ensheath the assist stent device 100), and to withdraw the capsule 703 proximally (to release the assist stent device 100).

The remainder of the delivery system 700 can be embodied using principles that are well-known in the art, and will not be described as this is well-known to a person skilled in the art.

In use, the cobalt-chromium assist stent device 100 is crimped on to the balloon 702. The sheath assembly is then advanced distally so that the capsule 703 completely covers the assist stent device 100.

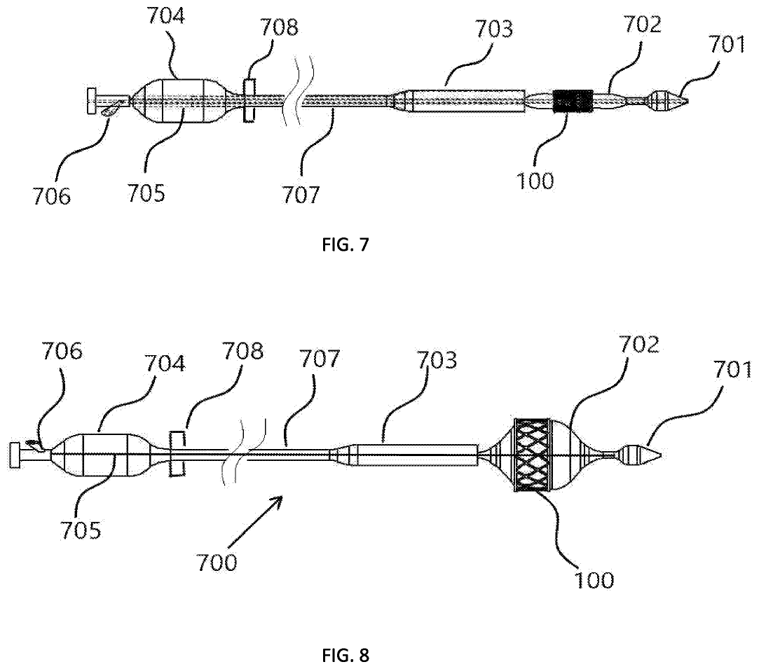

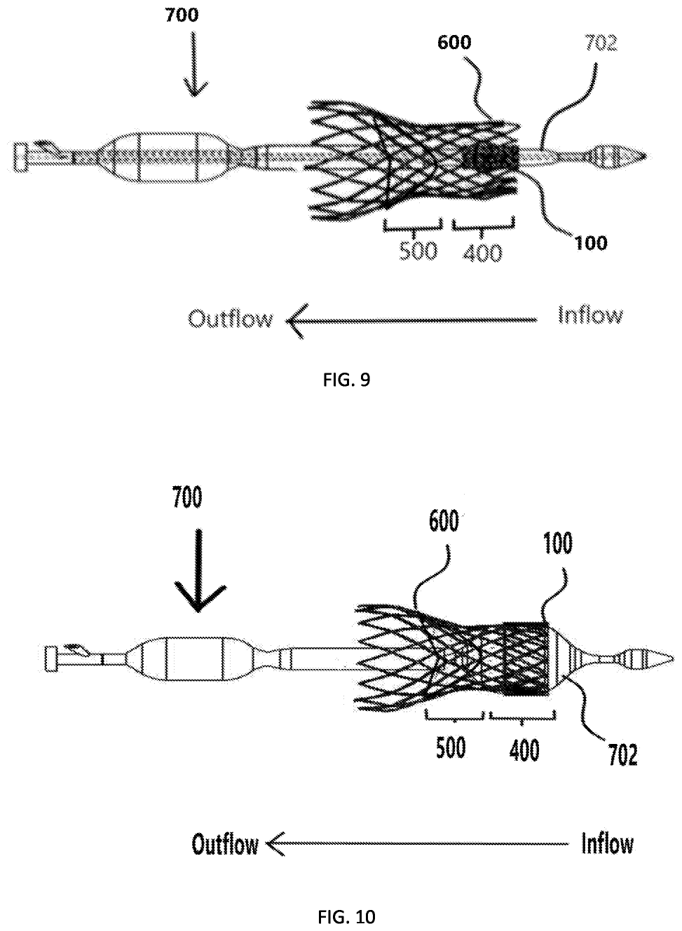

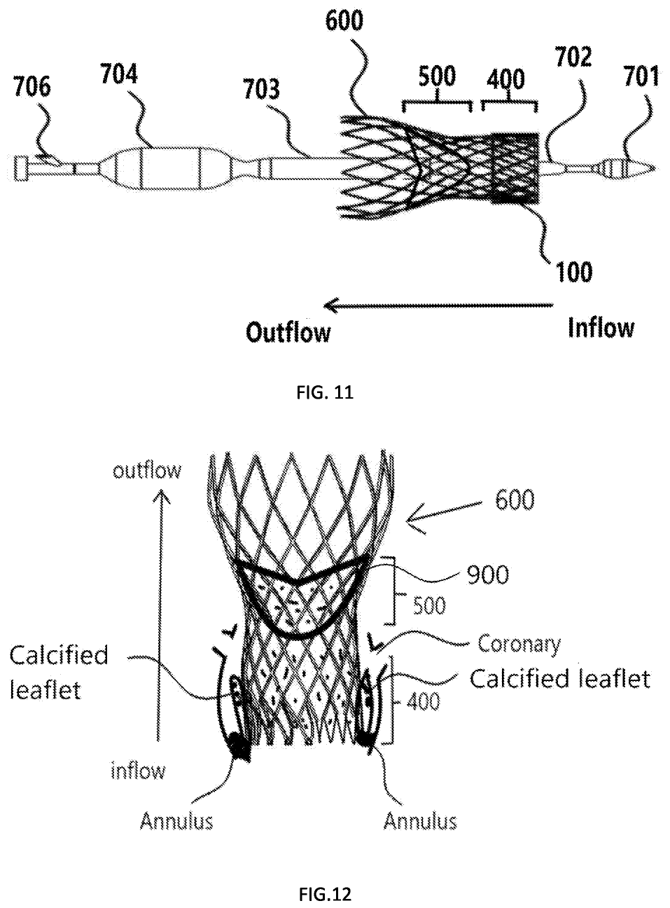

When the assist stent device 100 has been delivered to the location of a native aortic annulus inside a patient, the sheath assembly can be withdrawn so that the capsule 703 is withdrawn to expose the assist stent device 100. See FIG. 7. FIG. 9 shows the assist stent device 100 positioned within the skirt section 400 of the implanted aortic valve prosthesis 600. Next, the balloon 702 is inflated (FIG. 8) to expand the assist stent device 100. FIG. 10 shows the assist stent device 100 positioned expanded against the skirt section 400 of the implanted aortic valve prosthesis 600 to re-shape the deformed skirt section 400. The balloon 702 is then deflated (FIG. 11) so that the balloon catheter can also be withdrawn.

FIG. 13 shows the resulting implantation of the assist stent device 100 inside the skirt section 400 of the implanted aortic valve prosthesis 600. Because the skirt section 400 is usually made of a more flexible material such as nickel-titanium, deformation can occur. The assist stent device 100 is made from a much stiffer material, such as cobalt-chromium, so that it is able to re-shape and support the deformed skirt section 400 at the location of the aortic annulus. Thus, the assist stent device 100 and method of the present invention allows for an existing implanted aortic valve prosthesis 600 to be repaired, so as to avoid the need for replacing (or fracking) the implanted aortic valve prosthesis 600.

The present invention provides a number of unique features and benefits. First, the assist stent device 100 has a simple stent-like structure. Second, the cobalt-chromium assist stent device 100 assists the nickel-titanium frame to regain its initial profile to ensure optimized valve leaflet behavior with a good hemodynamics and no valve dysfunction, no regurgitation, no prolapse and no leaks.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A method for repairing an implanted heart valve prosthesis, wherein the heart valve prosthesis has a skirt section that is positioned at a native valve annulus, and a valve section which has a plurality of leaflets, comprising the steps of:
providing an assist stent device having an annular frame surrounded by an annular tissue assembly, wherein the annular frame has a generally cylindrical body made of cobalt chromium having a plurality of rows of cells;
delivering the assist stent device to the native valve annulus;
aligning the assist stent device inside the skirt section; and
expanding the assist stent device until the assist stent device engages the skirt section and secures the skirt section against the native valve annulus.

2. The method of claim 1, wherein the cylindrical body has two rows of diamond-shaped cells, and the cylindrical body has the same diameter throughout.

3. The method of claim 1, wherein the tissue assembly has an annular tissue that is stitched to the cylindrical body.
